(19) United States
(12) Patent Application Publication
(10) Pub. No.: US 2009/0157111 A1
(43) Pub. Date: Jun. 18, 2009

(54) End Cutting Vitrectomy Probe

(76) Inventors: Toh Seng Goh, Ballwin, MO (US); Paul Andrew Bennett, St. Louis, MO (US)

Correspondence Address: Bausch & Lomb Incorporated, One Bausch & Lomb Place, Rochester, NY, 14604-2701, US

(21) Appl. No.: 11/956,585

(22) Filed: Dec. 14, 2007

(51) Int. Cl. A61F 9/007

(52) U.S. Cl. 606/171

(57) ABSTRACT

An end-cutting vitrectomy probe 100 is provided that includes a hollow sleeve 102 having an opening 108 at the sleeve's distal end, and a tapered annular surface 112 disposed in the interior of the sleeve's distal end. The probe further includes a cutting member 120 disposed within the sleeve 102, and having a circumferential cutting edge 122 at its end. The cutting member 120 is moveable within the sleeve 102, such that the circumferential cutting edge 122 frictionally engages the tapered annular surface 112, to thereby cut any vitreous tissue disposed therebetween. The probe 100 may further include a drive mechanism 130 for slidably displacing the cutting member 120 within the hollow sleeve 102 in a reciprocating manner, to oscillate the cutting member 120 between engagement and disengagement with the tapered annular surface 112. The probe 100 may further include a pneumatic device 140 configured to apply a vacuum to the sleeve for aspirating vitreous through the opening 108 to be cut

[symbol]

### FIELD

The present invention is related to microsurgical probes and more specifically to ophthalmic microsurgical probes such as vitrectomy probes.

### BACKGROUND

The statements in this section merely provide background information related to the present disclosure and may not constitute prior art.

Ophthalmic surgical procedures on the posterior segment of the eye generally require the cutting and/or removal of the vitreous humor, a transparent jelly-like material that fills the posterior segment of the eye. The vitreous humor, or vitreous, is composed of numerous microscopic transparent fibers that are often attached to the retina. Therefore, cutting and removal of the vitreous must be done with great care to avoid traction on the retina, i.e., the separation of the retina from the choroid, a retinal tear, or, in the worst case, cutting and removal of the retina itself.

The use of microsurgical cutting probes in posterior segment ophthalmic surgery is well known. Such vitrectomy probes are typically inserted via an incision in the sclera near the pars plana. The surgeon may also insert other microsurgical instruments such as a fiber optic illuminator, an infusion cannula, or an aspiration probe during the posterior segment surgery. The surgeon may perform the surgical procedure while viewing the eye under a microscope.

Conventional vitrectomy probes typically include a hollow outer cutting member, a hollow inner cutting member arranged coaxially with and movably disposed within the hollow outer cutting member, and a port extending radially through the outer cutting member near the distal end thereof. Vitreous humor is aspirated into the open port, and the inner member is actuated, closing the port. Upon the closing of the port, cutting surfaces on both the inner and outer cutting members cooperate to cut the vitreous, and the cut vitreous is then aspirated away through the inner cutting member.

During posterior segment ophthalmic surgery, it is generally desirable to remove as much of the overlying vitreous as possible prior to any procedure to repair the underlying retina. However, a surgeon is limited in how close to the retina he or she can dispose a conventional vitrectomy probe due to both the probe's geometry and due to the risk of pulling or tearing vitreous strands near the retina that could lead to separation of the retina. Therefore, a need continues to exist for an improved vitrectomy probe that does not suffer from the above-described limitations.

### SUMMARY

In accordance with one aspect of the present application, an end-cutting vitrectomy probe is provided. The probe includes a hollow sleeve having an opening in the sleeve at its distal end portion, and a tapered annular surface disposed in the interior of the sleeve's distal end. The end-cutting vitrectomy probe further includes a cutting member having a distal end defining a circumferential cutting edge, where the cutting member is slidably disposed within the hollow sleeve. The cutting member is movable towards the distal end of the sleeve, such that the circumferential cutting edge frictionally engages the tapered annular surface within the hollow sleeve, to thereby cut vitreous tissue disposed therebetween. The end-cutting vitrectomy probe may further include a drive mechanism for slidably displacing the cutting member within the hollow sleeve in a reciprocating manner, such that the cutting member oscillates between a position of engagement and disengagement with the tapered annular surface. The end cutting vitrectomy probe may further include a pneumatic device configured to apply a vacuum to the interior of the hollow sleeve, for aspirating vitreous tissues in through the opening such that portions of the vitreous tissues may be cut.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 shows several views of an end portion of an end-cutting vitrectomy probe, in various positions of movement in accordance with the present invention;

FIG. 2 is an exploded view of a portion of an end-cutting vitrectomy probe in accordance with one aspect of the present application;

FIG. 3 is a section view of one embodiment of an end-cutting vitrectomy probe in accordance with one aspect of the present application;

FIG. 4 is a section view of a second embodiment of an end-cutting vitrectomy probe in accordance with one aspect of the present application; and

FIG. 5 is a sectional view of an alternate embodiment of an end-cutting vitrectomy probe having a drive mechanism and a cam-follower for inducing rotation of a cutting member in accordance with one aspect of the present application.

### DETAILED DESCRIPTION

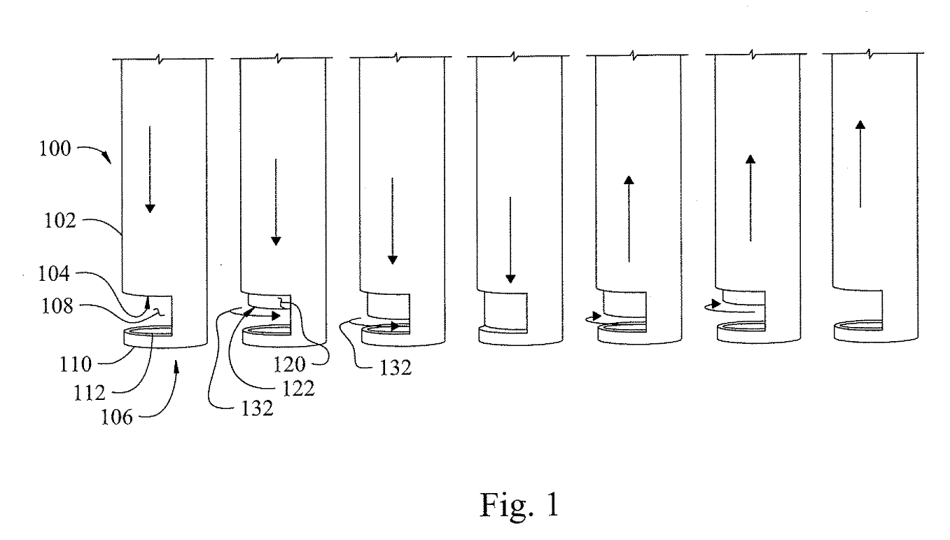

The following description is merely exemplary in nature and is not intended to limit the present disclosure, application, or uses. Various embodiments and their advantages are best understood by referring to FIGS. 1 through 5 of the drawings, like numerals being used for like and corresponding parts of the various drawings.

FIG. 1 shows the distal portion of a vitrectomy probe 100 according to one preferred embodiment. Probe 100 generally includes an outer tubular body or hollow sleeve 102 having an inner bore 104, a closed distal end or tip 106, and an opening or notch 108 providing access to the interior or inner bore 104. The hollow sleeve or tubular body 102 is preferably made of stainless steel or other suitable material. An inner cutting member 120 longitudinally reciprocates within the tubular body 102 so as to cut tissue, which is aspirated into the inner bore and though a remote port (not shown). The inner cutting member 120 may also be comprised of a hollow sleeve or tube body.

It is essential to ensure that proper cutting of vitreous between the cutting member and end of the sleeve occurs. This is critical, since vitreous tissue strands near the retina must be cut cleanly to avoid pulling of vitreous strands that could cause pulling the retina away from the eye wall. Accordingly, one important aspect of the present application addresses the engagement between the cutting member and a cutting block, plate or disc element, to provide for alignment of the cutting member that ensures adequate cutting engagement for cleanly cutting vitreous tissues.

As shown in FIG. 1, one embodiment of an end-cutting vitrectomy probe 100 is provided that comprises a hollow sleeve 102 having an inner bore 104 extending to a distal end 106. The distal end portion of the sleeve 102 has an opening or notch 108 therein, and a closed or distal end 106 has a tapered annular surface 112 disposed within the interior of the sleeve 102. The end-cutting vitrectomy probe 100 further includes an inner cutting member 120 having a distal end defining a circumferential cutting edge 122, where the cutting member 120 is slidably disposed within the hollow sleeve 102. The cutting member 120 is movable towards the distal end 106 of the sleeve 102, such that the circumferential cutting edge 122 frictionally engages the tapered annular surface 112 within the hollow sleeve 102, to thereby cut any vitreous tissue disposed between the circumferential cutting edge 122 and the tapered annular surface 112. The end-cutting vitrectomy probe 100 may further include a drive mechanism 130 for slidably displacing the cutting member 120 within the hollow sleeve 102 in a reciprocating manner, such that the cutting member 120 oscillates between a position of engagement with the tapered annular surface 112 and a position spaced apart from the tapered annular surface 112, Accordingly, the drive mechanism thereby provides for repetitive cutting action with the cutting member. The end cutting vitrectomy probe may further include a pneumatic device 140 configured to apply a vacuum to the interior 104 of the hollow sleeve 102, for aspirating vitreous tissues in through the opening 108 and into the interior of the hollow sleeve 104. This introduces portions of the vitreous tissues into the sleeve 102, which may be cut and drawn through the interior of the hollow sleeve 102 and/or cutting member 120.

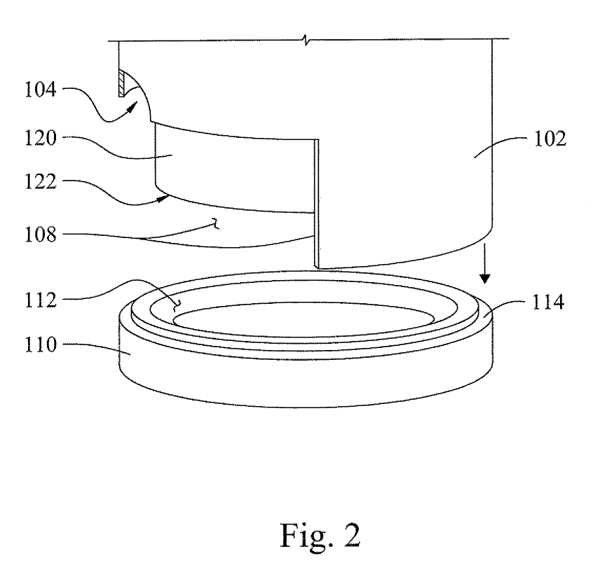

Referring to FIG. 2, the tapered annular surface 112 may be formed on a disc element 110, which is preferably secured to the distal end of the hollow sleeve 102. The disc element 110 may be secured to the distal end by means of welding, ultrasonic welding, crimping, adhesive bonding, or any other suitable securing means. The disc element 110 is secured on the distal end of the hollow sleeve 102 with the tapered annular surface 112 facing the interior 104 of the sleeve's distal end. The disc element 110 may further include an outer annular shoulder 114 for assisting the fit or assembly of the disc element 110 onto the distal end of the hollow sleeve 102.

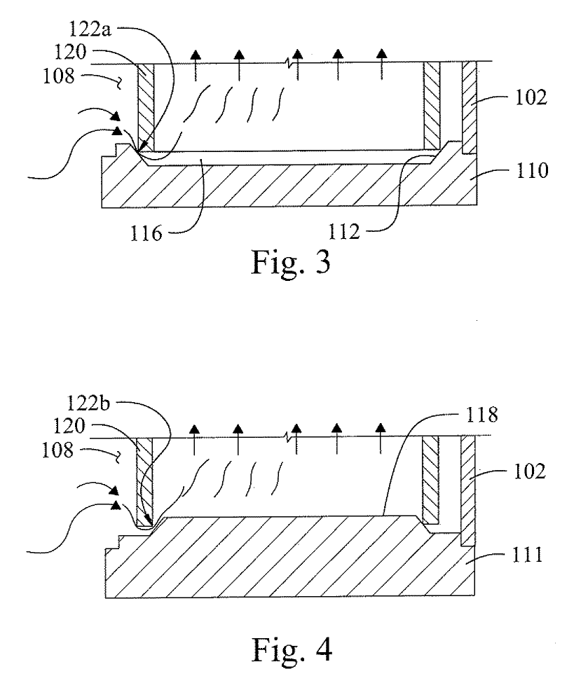

Referring to FIG. 3, the tapered annular surface 112 on the disc element 110 may form part of a recess 116 in the disc element 110. Accordingly, cutting of vitreous drawn into the sleeve 102 may occur when the outer circumferential cutting edge 122a of the cutting member 120 strikes the slanted edge of the tapered annular surface 112 of the recess 116 formed in the disc element 110.

Alternatively, the tapered annular surface on the disc element may form part of a raised portion 118 on a disc element 111, as shown in FIG. 4. Accordingly, cutting of vitreous drawn into the sleeve 102 may occur when the inner circumferential cutting edge 122b of the cutting member 120 strikes the slanted edge of the tapered annular surface 112 forming part of the raised portion 118 on the disc element 111.

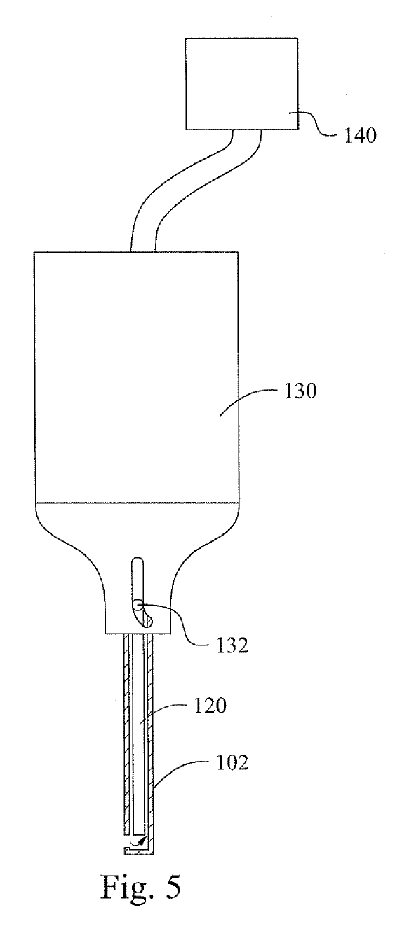

The drive mechanism 130 may further be configured to rotate the cutting member 120 within the sleeve 102, as shown by arrow 132 in FIG. 1. Preferably, the drive mechanism is configured to rotate the cutting member 120 in a first rotation direction while the cutting member 120 is moved towards the sleeve's distal end 106, and to rotate the cutting member 120 in a second rotation direction opposite the first direction while the cutting member 120 is moved away from the sleeve's distal end 106. As shown in FIG. 5, a motor 140 and drive mechanism 130 could further incorporate a cam-follower structure 132 or other known structure for inducing rotation during reciprocation of cutting member 120 within sleeve 102.

When the cutting member 120 is moved toward the distal end of the sleeve 102 into a position of contact with the disc element 110, it is essential to ensure that proper engagement between the circumferential cutting edge 122 and the disc 110 occurs. This is critical, since vitreous near the retina must be cut cleanly to avoid pulling of vitreous strands that could cause pulling the retina away from the eye wall. Accordingly, one important aspect of the tapered annular surface 112 on the disc element 110 is to provide for alignment of the cutting member 120 to ensure adequate cutting engagement for cleanly cutting vitreous. Upon contact between the cutting member 120 and the tapered annular surface 112, the tapered annular surface 112 guides or adjusts the circumferential cutting edge 122, to concentrically align the end of the cutting member 120 with the tapered annular surface 112. This alignment ensures that a substantial portion of the circumference of the cutting edge 122 engages the tapered annular surface 112, to thereby provide for a clean cut of any vitreous between the cutting edge 122 and the disc's tapered annular surface 112.

It should be noted that some embodiments may include only the sleeve 102, disc 110 and cutting element 120 as part of a disposable, easily-replaced component, for use with any number of end-cutting vitrectomy probe systems. Alternatively, other embodiments may include one or more of the above mentioned elements. In a second embodiment, an end-cutting vitrectomy probe assembly is provided that includes a cutting member within a hollow sleeve having an opening in a side wall of the sleeve's distal end portion, and a tapered annular surface disposed in the interior of the sleeve's distal end. The cutting member is movably disposed within the hollow sleeve, and has a cylindrical distal end defining a circumferential cutting edge. When the cutting member is fully displaced against the distal end of the sleeve, it frictionally engages the tapered annular surface within the hollow sleeve, to thereby cut any vitreous tissue disposed between the circumferential cutting edge and the tapered annular surface. The end-cutting vitrectomy probe assembly further includes a pneumatic device configured to apply a vacuum to the interior of the hollow sleeve, for aspirating vitreous tissues in through the opening and into the interior of the hollow sleeve, such that portions of the vitreous tissues may be drawn through the opening and cut. The end-cutting vitrectomy probe assembly further includes a drive mechanism for slidably displacing the cutting member within the hollow sleeve in a reciprocating manner. This permits the cutting member to oscillate between a position of engagement with the tapered annular surface, and a position spaced apart from the tapered annular surface, to thereby provide for repetitive cutting action.

In another aspect of the present application, the end-cutting vitrectomy probe is further configured to provide for alignment of the cutting member to ensure adequate cutting engagement for cleaning cutting vitreous tissues. The drive mechanism may be configured to rotate the cutting member in a first rotation direction as shown in FIG. 1, while the cutting member 120 is moving towards the sleeve's distal end 106. Upon contact between the cutting member 120 and the disc 110 having a tapered annular surface, the disc's tapered annular surface guides or adjusts the circumferential cutting edge 122, to concentrically align the end of the cutting member 120 with the tapered annular surface. Specifically, the rotational contact of the circumferential cutting edge 122 against the tapered annular surface 112 causes the circumferential cutting edge 122 to concentrically align with the tapered annular surface 112, to cause a substantial portion of the circumference of the cutting edge 122 to engage the tapered annular surface 112 and thereby improve cutting of any vitreous tissue therebetween. After a period of engagement between the cutting member 120 and disc 110, the drive mechanism is configured to rotate the cutting member 120 in a second rotation direction opposite the first direction. The drive mechanism continues to rotate in the second rotational direction while the cutting member 120 is moving away from the sleeve's distal end 106. The above concentric alignment ensures that a substantial portion of the circumference of the cutting edge 122 engages the tapered annular surface 112, to thereby provide for a clean cut of any vitreous tissues between the cutting edge and the tapered annular surface.

From the above, it may be appreciated that the present invention provides improved apparatus and methods of performing vitrectomy surgery. The present invention is illustrated herein by example, and various modifications may be made by a person of ordinary skill in the art.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. An end-cutting vitrectomy probe, comprising:
a hollow sleeve having an opening in the sleeve at its distal end portion, and a tapered annular surface disposed in the interior of the sleeve's distal end;
a cutting member slidably disposed within the hollow sleeve and having a distal end defining a circumferential cutting edge, the cutting member being movable towards the distal end of the sleeve such that the circumferential cutting edge frictionally engages the tapered annular surface within the hollow sleeve, to thereby cut any vitreous tissue disposed between the circumferential cutting edge and the tapered annular surface.

2. The end-cutting vitrectomy probe of claim 1 further comprising a drive mechanism for slidably displacing the cutting member within the hollow sleeve in a reciprocating manner, such that the cutting member oscillates between a position of engagement with the tapered annular surface and a position spaced apart from the tapered annular surface, to thereby provide for repetitive cutting action.

3. The end cutting vitrectomy probe of claim 2 further comprising a pneumatic device configured to apply a vacuum to the interior of the hollow sleeve, for aspirating vitreous tissues in through the opening into the interior of the hollow sleeve.

4. The end-cutting vitrectomy probe of claim 1 wherein the tapered annular surface is formed on a disc element, which is secured on the distal end of the hollow sleeve with the tapered annular surface facing the interior of the sleeve's distal end.

5. The end-cutting vitrectomy probe of claim 4 wherein the tapered annular surface forms part of a recess in the disc element.

6. The end-cutting vitrectomy probe of claim 4 wherein the tapered annular surface forms part of a raised portion on the disc element.

7. The end-cutting vitrectomy probe of claim 2 wherein the drive mechanism is further configured to rotate the cutting member.

8. The end-cutting vitrectomy probe of claim 2 wherein the drive mechanism is further configured to rotate the cutting member in a first rotation direction while the cutting member is moved towards the sleeve's distal end, and to rotate the cutting member in a second rotation direction opposite the first direction while the cutting member is moved away from the sleeve's distal end.

9. The end-cutting vitrectomy probe of claim 4, wherein upon contact between the cutting member and the tapered annular surface, the tapered annular surface guides the circumferential cutting edge to concentrically align the end of the cutting member with the tapered annular surface, such that a substantial portion of the circumference of the cutting edge engages the tapered annular surface.

10. An end-cutting vitrectomy probe, comprising:
a hollow sleeve with a distal end portion and having an opening in a side wall of the sleeve's distal end portion, and a tapered annular surface disposed in the interior of the sleeve's distal end;
a pneumatic device configured to apply a vacuum to the interior of the hollow sleeve, for aspirating vitreous tissues in through said opening into the interior of the hollow sleeve;
a cutting member movably disposed within the hollow sleeve, the cutting member having a cylindrical distal end defining a circumferential cutting edge which, when the cutting member is fully displaced against the distal end of the sleeve, frictionally engages the tapered annular surface within the hollow sleeve, to thereby cut any vitreous tissue disposed between the circumferential cutting edge and the tapered annular surface; and
a drive mechanism for slidably displacing the cutting member within the hollow sleeve in a reciprocating manner, such that the cutting member oscillates between a position of engagement with the tapered annular surface and a position spaced apart from the tapered annular surface, to thereby provide for repetitive cutting action.

11. The end-cutting vitrectomy probe of claim 10 wherein the tapered annular surface is formed on a disc element, which is secured on the distal end of the hollow sleeve with the tapered annular surface facing the interior of the sleeve's distal end.

12. The end-cutting vitrectomy probe of claim 11 wherein the tapered annular surface forms part of a recess in the disc element.

13. The end-cutting vitrectomy probe of claim 11 wherein the tapered annular surface forms part of a raised portion on the disc element.

14. The end-cutting vitrectomy probe of claim 11 wherein the drive mechanism is further configured to rotate the cutting member.

15. The end-cutting vitrectomy probe of claim 11 wherein the drive mechanism is further configured to rotate the cutting member in a first rotation direction while the cutting member is moved towards the sleeve's distal end, and to rotate the cutting member in a second rotation direction opposite the first direction while the cutting member is moved away from the sleeve's distal end.

16. The end-cutting vitrectomy probe of claim 15, wherein upon contact between the cutting member and the tapered annular surface, the tapered annular surface guides the circumferential cutting edge to concentrically align the end of the cutting member with the tapered annular surface, such that a substantial portion of the circumference of the cutting edge engages the tapered annular surface.

17. An end-cutting vitrectomy probe, comprising:
a hollow sleeve having an opening in a side wall of the sleeve's distal end portion;
a disc element having a tapered annular surface on one face, the disc element being secured on the distal end of the hollow sleeve with the tapered annular surface facing the interior of the sleeve's distal end;
a pneumatic device configured to apply a vacuum to the interior of the hollow sleeve, for aspirating vitreous tissues in through said opening into the interior of the hollow sleeve;
a cutting member movably disposed within the hollow sleeve, the cutting member having a cylindrical distal end defining a circumferential cutting edge which, when the cutting member is fully displaced against the distal end of the sleeve, frictionally engages the tapered annular surface within the hollow sleeve, to thereby cut any vitreous tissue disposed between the circumferential cutting edge and the tapered annular surface; and
a drive mechanism for slidably displacing the cutting member within the hollow sleeve in a reciprocating manner, such that the cutting member oscillates between a position of engagement with the tapered annular surface and a position spaced apart from the tapered annular surface, to thereby provide for repetitive cutting action.

18. The end-cutting vitrectomy probe of claim 17 wherein the tapered annular surface forms part of a recess in the disc element.

19. The end-cutting vitrectomy probe of claim 17 wherein the tapered annular surface forms part of a raised portion on the disc element.

20. The end-cutting vitrectomy probe of claim 11 wherein the drive mechanism is further configured to rotate the cutting member in a first rotation direction while the cutting member is moving towards the sleeve's distal end, such that rotational contact of the circumferential cutting edge against the tapered annular surface causes the circumferential cutting edge to concentrically align with the tapered annular surface to cause a substantial portion of the circumference of the cutting edge to engage the tapered annular surface and thereby cut any vitreous tissue therebetween, after which the drive mechanism is configured to rotate the cutting member in a second rotation direction opposite the first direction while the cutting member is moving away from the sleeve's distal end.
